(19) United States
(12) Patent Application Publication
(10) Pub. No.: US 2010/0305633 A1
(43) Pub. Date: Dec. 2, 2010

(54) Diagnostic And Therapeutic Chest Casing

(76) Inventor: Kusai Saadeldin Aziz, Visalia, CA (US)

Correspondence Address: Ashok Tankha, 36 Greenleigh Drive, Sewell, NJ, 08080, US

(21) Appl. No.: 12/791,007

(22) Filed: Jun. 1, 2010

Related U.S. Application Data
(60) Provisional application No. 61/183,085, filed on Jun. 2, 2009.

(51) Int. Cl. A61N 1/00; A61B 5/0205; A61F 7/00; A61B 5/00; A61M 16/00

(52) U.S. Cl. 607/3; 600/484; 607/96; 600/301; 128/204.21

(57) ABSTRACT

A system and method is provided for detection, forewarning, and rapid therapeutic treatment of a patient's ischemic and arrhythmic heart condition, congestive heart failure, respiratory failure, etc. The system comprises a chest casing of a form fitting material. The inner surface of the chest casing comprises electrocardiogram sensor electrodes, auscultation sensors, impedance sensor electrodes, etc. The electrocardiogram sensor electrodes detect abnormal electrocardiogram signals. The auscultation sensors record internal sounds at predefined cardiac and respiratory auscultation sensor points. The impedance sensor electrodes measure thoracic impedance across two or more points on the chest wall. A control unit is connected to the sensors for collecting and processing patient information. The control unit transmits the processed patient information to an external monitoring station. The chest casing comprises therapeutic delivery points for delivering therapeutic electrical dosages and different therapies, for example, cooling therapy, to the patient via therapeutic devices connected on the chest casing.

### CROSS REFERENCE TO RELATED APPLICATIONS

This application claims the benefit of provisional patent application No. 61/183,085 titled “Cardiac Diagnostic and Therapeutic Chest Casing”, filed on Jun. 2, 2009 in the United States Patent and Trademark Office.

The specification of the above referenced patent application is incorporated herein by reference in its entirety.

### BACKGROUND

The incidence of coronary artery diseases is increasing worldwide. Myocardial infarction, commonly known as heart attack, has been a major cause of death in most countries. A large number of patients suffer death or irreparable damage to heart muscles due to failure in obtaining immediate medical attention. Therefore, early recognition of myocardial infarction is critical in saving a life and preventing or limiting damage to the heart muscles according to the adage “time is muscle”. A combination of signs, symptoms and electrocardiogram (ECG) diagnoses is known to be associated with this heart condition, and can be used to detect and prevent incidents of major damage due to congestive heart failure, heart attacks, and cardiac arrhythmias.

Hence, there is a long felt but unresolved need for a system and method for early detection, diagnosis, and automated rapid therapeutic treatment of ischemic and arrhythmic heart conditions, and other serious cardiopulmonary conditions in a patient, and for alerting the patient, a health care provider, and emergency medical services for immediate medical attention.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The system and method disclosed herein addresses the above stated need for preventing incidents of major damage due to ischemic and arrhythmic heart conditions by addressing early detection, forewarning, and automated rapid therapeutic treatment of ischemic and/or arrhythmic heart conditions and other life threatening cardiopulmonary conditions in a patient. The system and method disclosed herein also alerts the patient, a health care provider, and emergency medical services for immediate medical attention.

The system disclosed herein comprises a chest casing made of a form fitting material configured to conform to an upper body of a patient. The chest casing defines an inner surface and an outer surface, opposite to each other. The inner surface establishes adequate contact with the skin of the patient by conforming to the contours of the upper body of the patient. The inner surface of the chest casing comprises a predefined number of electrocardiogram sensor electrodes and a predefined number of auscultation sensors. The electrocardiogram sensor electrodes detect abnormal electrocardiogram signals of the patient. The auscultation sensors record internal sounds at predefined cardiac auscultation sensor points and respiratory auscultation sensor points located on a chest wall of the patient. The predefined cardiac auscultation sensor points on the chest wall of the patient for recording the internal sounds by the auscultation sensors correspond to the apex of the heart, the aortic valve area, and the left sternal border. The respiratory auscultation sensor points on the chest wall of the patient for recording the internal respiratory sounds by the auscultation sensors correspond to the lower lung fields, the middle lung fields, and the upper lung fields on a back of the patient. The auscultation sensors are therefore positioned on the inner surface of the chest casing corresponding to the apex of heart, the aortic valve area, the left sternal border, the lower lung fields, the middle lung fields, and the upper lung fields on the patient's back for recording the internal sounds.

The chest casing further comprises one or more sleeves made of the form fitting material configured to conform to the shoulders and arms of the patient. The inner surface of the sleeves comprises one or more sensors, for example, a temperature sensor, a pulse oxygenation sensor, a blood pressure sensor, etc.

The system disclosed herein further comprises a control unit attached to the outer surface of the chest casing and connected to the electrocardiogram sensor electrodes and the auscultation sensors for collecting and processing patient information comprising one or more signals from the electrocardiogram sensor electrodes and the auscultation sensors. The control unit also communicates with an external monitoring station for transmitting the processed patient information to the external monitoring station. The external monitoring station communicates with emergency medical services for alerting the emergency medical services of an emergency based on the transmitted patient information. The chest casing further comprises one or more therapeutic delivery points for delivering therapeutic electrical dosages to the patient automatically and/or based on feedback from the external monitoring station to the control unit in response to the patient information received by the external monitoring station. The chest casing can be used as part of a telemedicine system or a stand alone system for other applications.

In an embodiment, the inner surface of the chest casing further comprises one or more impedance sensor electrodes for measuring thoracic impedance across two or more points on the chest wall of the patient. The control unit processes the measured thoracic impedance to detect signs of abnormality between the two or more points, to detect congestive heart failure, etc. The chest casing further comprises one or more embedded coolant circuits for delivering cooling therapy to lower the body temperature of the patient. The system disclosed herein further comprises chest wall pads positioned on the chest casing for shock delivery. A defibrillator unit and/or a pacemaker connected to the chest wall pads automatically diagnose curable rhythms and deliver the therapeutic electrical dosages to the patient to restore normal sinus rhythm.

The control unit further comprises a user interface for enabling the patient to enter patient information, demographic information, medical condition information, etc. The patient may also receive instructions and feedback from a physician or other health care provider at the external monitoring station via the user interface. The patient may also send an alert to the external monitoring station via the user interface.

The control unit measures and stores baseline patient information comprising, for example, baseline electrocardiogram, baseline heart sounds, baseline lung sounds, baseline heart rate, blood pressure, temperature, oxygen saturation, baseline impedance, etc. The control unit detects abnormal variations in the heart and cardiopulmonary conditions of the patient by comparing the baseline patient information with the collected patient information.

The control unit attached to the chest casing dynamically adjusts the automated delivery of one or more of the therapeutic electrical dosages, the cooling therapy, and other therapies, in response to changes in the collected patient information, the heart and cardiopulmonary conditions, and/or instructions and feedback received from the external monitoring station. For example, the control unit may progressively or gradually adjust the delivery of the therapeutic electrical dosages in a form of shock or externally paced beats and the cooling therapy using the therapeutic devices in response to changes in a subsequent batch of the patient information, which may indicate the patient's response to the ongoing therapeutic electrical dosages and/or the cooling therapy. The system disclosed herein further comprises one or more therapeutic devices positioned on the chest casing and/or in the vicinity of the patient and controlled by the control unit for delivering multiple therapies to the patient. For example, the system disclosed herein further comprises an oxygen storage unit and an oxygen delivery apparatus positioned on the chest casing for delivering oxygen to the patient. The control unit controls the delivery of the oxygen by the oxygen storage unit and the oxygen delivery apparatus based on one or more of the collected patient information, the feedback from the external monitoring station, online data measured on the patient, for example, the impedance across two points on the chest wall of the patient, etc. The control unit further comprises a global positioning system for transmitting information on location of the patient to the external monitoring station for alerting emergency medical services (EMSs).

The method and system disclosed herein collects symptoms entered by the patient, electrocardiogram (ECG) diagnosis, and other physical diagnoses, and automatically detects abnormalities associated with acute myocardial infarction or life threatening rhythms. The method and system disclosed herein enables early diagnosis of urgent and life threatening cardiopulmonary emergencies such as myocardial infarction or heart attacks, arrhythmia, for example, very fast or slow heart rhythm, congestive heart failure, respiratory failure or a patient's inability to breath, etc. The system disclosed herein automatically activates emergency medical services, forwards the collected patient information to an external monitoring station where further analyses may be performed, and alerts the patient, emergency medical services (EMSs) or health care centers about any life threatening conditions. Though the method and system disclosed herein is described with respect to detecting myocardial infarctions and arrhythmias, the method and system disclosed herein can be used to diagnose and treat a variety of medical conditions and monitor the general well being of the patient. For example, the system disclosed herein can be used in the diagnosis and treatment of other serious cardiopulmonary conditions such as congestive heart failure and respiratory failure.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and instrumentalities disclosed herein.

FIG. 1 exemplarily illustrates a system for detection, forewarning, and rapid therapeutic treatment of heart and cardiopulmonary conditions in a patient.

FIG. 2A exemplarily illustrates an inner surface view of the front of a chest casing.

FIG. 2B exemplarily illustrates an inner surface view of the back of a chest casing.

FIG. 2C exemplarily illustrates a front view of an outer surface of the chest casing.

FIG. 3 illustrates a method for detection, forewarning, and rapid therapeutic treatment of heart and cardiopulmonary conditions in the patient.

FIG. 4 exemplarily illustrates a flow diagram for detection, forewarning, and therapeutic treatment of a heart condition in the patient.

FIG. 5 exemplarily illustrates an architectural block diagram of the system for detection, forewarning, and rapid therapeutic treatment of heart and cardiopulmonary conditions in a patient.

### DETAILED DESCRIPTION OF THE INVENTION

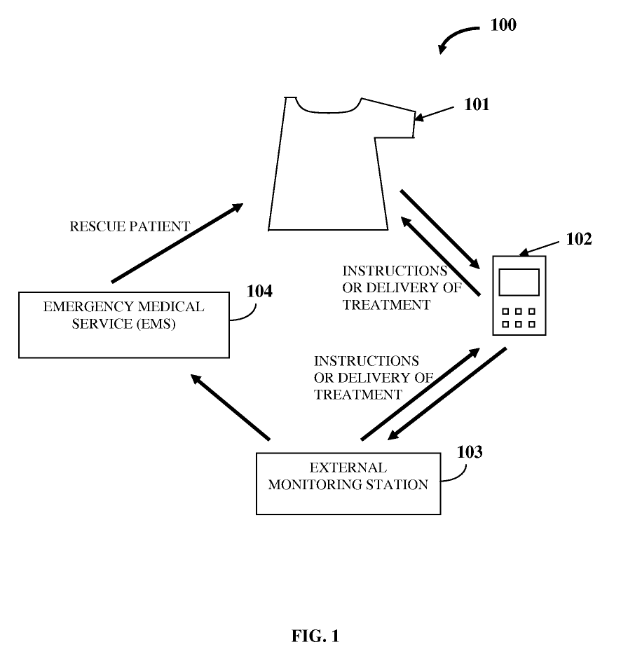

FIG. 1 exemplarily illustrates a system 100 for detection, forewarning, and rapid therapeutic treatment of heart and cardiopulmonary conditions, for example, an ischemic and arrhythmic heart condition, congestive heart failure, respiratory failure, etc. in a patient. Ischemic heart conditions leading to myocardial infarction are primarily detected using electrocardiograms (ECGs). Severe arrhythmic heart conditions such as ventricular tachycardia, ventricular fibrillation, severe bradycardia and other forms of tachy and brady arrhythmias are detected using simple auscultation techniques and by monitoring cardiac cycles from the ECG. The system 100 disclosed herein comprises a chest casing 101 made of a form fitting material, for example, an elastomeric material, configured to conform to an upper body of the patient. As used herein, the “chest casing” refers to a wearable casing, for example, in the form of a wearable jacket, belt, or any other wearable garment or apparel providing firm contact with the skin of the patient. The chest casing 101 defines an inner surface 101a and an outer surface 101b, each opposite to the other. The inner surface 101a establishes adequate contact with the skin of the patient by conforming to the contours of the upper body of the patient.

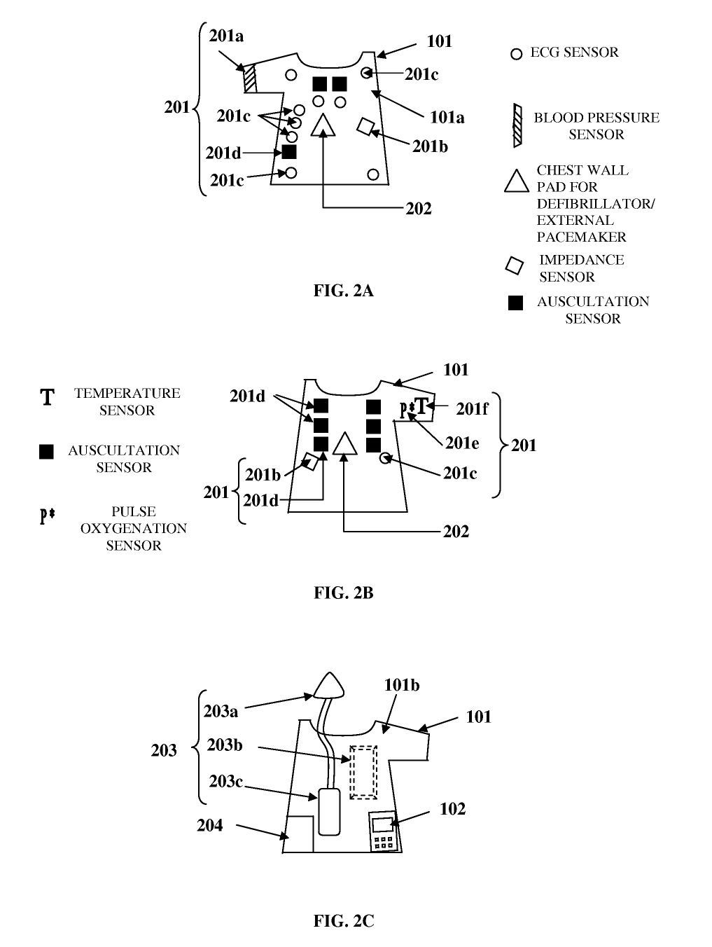

The inner surface 101a of the chest casing 101 comprises a predefined number of electrocardiogram (ECG) sensor electrodes 201c for detecting abnormal electrocardiogram (ECG) signals of the patient. The detection of an abnormal condition occurs when the electrocardiogram signal, for example, registers irregular cardiac cycles or frequent abnormalities in the cardiac cycle for a specified period of time. The placement of ECG sensor electrodes 201c or ECG leads is according to the standard placement of ECG leads for optimum recording of electrical signals of the heart. For example, the placement of ECG sensor electrodes 201c on the front and back inner surfaces 101a of the chest casing 101 as exemplarily illustrated in FIG. 2A and FIG. 2B respectively.

Conditions such as congestive heart failure and respiratory failure can be detected by measuring and analyzing heart sounds and breathing sounds. Accordingly, the inner surface 101a of the chest casing 101 further comprises a predefined number of auscultation sensors 201d for recording internal sounds at predefined cardiac auscultation sensor points and respiratory auscultation sensor points located on the chest wall of the patient. The cardiac auscultation sensor points on the chest wall comprise, for example, locations corresponding to the apex of the heart, the aortic valve area, and the left sternal border. The respiratory auscultation sensor points on the chest wall of the patient correspond to, for example, lower lung fields, middle lung fields, and upper lung fields on the patient's back. For example, the auscultation sensors 201d are positioned on the inner surface 101a of the chest casing 101 as exemplarily illustrated in FIG. 2B.

In an embodiment, the inner surface 101a of the chest casing 101 comprises one or more impedance sensor electrodes 201b for measuring thoracic impedance across two or more points on the chest wall of the patient. The positioning of the impedance sensor electrodes 201b on the front and back inner surfaces 101a of the chest casing 101 is exemplarily illustrated in FIGS. 2A-2B. A control unit 102 attached to the outer surface 101b of the chest casing 101 and in electronic communication with the impedance sensor electrodes 201b processes the measured thoracic impedance to detect signs of abnormality between the two or more points, to detect signs of congestive heart failure, etc. In case of a congestive heart failure, body organs such the lungs become congested with fluid. Detection of congestive heart failure is based on the principle that the body's impedance can be lower in areas of higher fluid build up. If the fluids in the lungs increase, the thoracic impedance across the two or more points on the chest wall decreases. Accordingly, the thoracic impedance can be used to measure the fluid content of the lungs and hence aid in the diagnosis of congestive heart failure. The impedance sensor electrodes 201b are, for example, electrodes interfacing with the skin of the patient and are configured to measure and generate impedance signals over a length of the cardiac cycle or over an extended period of time. The control unit 102 controls a therapeutic device that administers or delivers medications to regulate, for example, increase thoracic impedance and to treat congestive heart failure.

The chest casing 101 further comprises one or more embedded coolant circuits 203b, as exemplarily illustrated in FIG. 2C, for delivering cooling therapy to regulate, for example, lower the body temperature of the patient. FIG. 2C exemplarily illustrates a front view of the outer surface 101b of the chest casing 101. Cooling therapy may be used to limit the damage of the heart muscle and reduce the impact on vital organs during emergency cardiac conditions. The embedded coolant circuits 203b may be activated via the control unit 102 for controlled circulation of a coolant within the coolant circuits 203b for even distribution of the coolant through the chest casing 101. The control unit 102 may control the rate of coolant circulation through the embedded coolant circuits 203b. The chest casing 101 further comprises chest wall pads 202, as exemplarily illustrated in FIGS. 2A-2B, interchangeably connected to a defibrillator unit 203d or an external pacemaker 203e depending on the patient's history of cardiac arrhythmia and present cardiac condition and requirement.

In an embodiment, the chest casing 101 comprises one or more sleeves made of the form fitting material configured to conform to the shoulders and arms of the patient. In an embodiment, the inner surface 101a of the sleeves comprises sensors 201, for example, a temperature sensor 201f, a pulse oxygenation sensor 201e, a blood pressure sensor 201a, ECG sensor electrodes 201c, etc. Each of the sensors 201 is connected to the control unit 102. The placement of these sensors 201 is exemplarily illustrated in FIGS. 2A-2B. Conditions such as low oxygenation of the body, that is, hypoxia, caused due to respiratory failure may be indicated using the pulse oxygenation sensor 201e.

The control unit 102 attached to the outer surface 101b of the chest casing 101 and connected to the sensors 201, for example, the electrocardiogram sensor electrodes 201c, the auscultation sensors 201d, the impedance sensor electrodes 201b, and other sensors, collects and processes patient information comprising one or more signals from the sensors 201. The control unit 102 can be a separate independent module or an integral part of the chest casing 101, as exemplarily illustrated in the front view of the outer surface 101b of the chest casing 101 in FIG. 2C. The collected patient information comprises one or more sensor signals, personal information of the patient, and patient history. For example, the personal information and patient history as entered beforehand through a user interface 102d of the control unit 102, comprise, for example, the name of the patient, age, gender, address, demographic information, medical condition, medications, and other information entered at earlier stages. Patient history comprises, for example, the history of cardiac conditions of the patient, the history of hospital admissions, the treating physicians or health care providers, etc.

The control unit 102 comprises a user interface 102d, as exemplarily illustrated in FIG. 5, for enabling the patient to enter patient information such as personal information, demographic information, and medical condition information and for receiving instructions and feedback from a physician or health care provider at the external monitoring station 103. The patient also uses the user interface 102d to describe the symptoms of the patient to a physician or health care provider at the external monitoring station 103. For example, the patient may press specific buttons assigned to describe physical symptoms such as chest pain, shortness of breath, heart racing, dizziness, feeling unwell, etc. on the user interface 102d based on the patient's condition. The patient also uses the user interface 102d to send an alert to the external monitoring station 103 or to communicate with the physician or health care provider at the external monitoring station 103. A power source 204, for example, a rechargeable battery is removably connected to the chest casing 101 and supplies power to the control unit 102 and the different sensors 201 and electrodes disposed on the chest casing 101. For example, the power source 204 supplies a low-amplitude current across the impedance sensor electrodes 201b for measuring the thoracic impedance.

The control unit 102 communicates with an external monitoring station 103 and/or an emergency medical service (EMS) 104 based on preset criteria or when the patient chooses to signal an emergency situation by pressing an emergency button on the user interface 102d of the control unit 102. The preset criteria for communicating with the external monitoring station 103 comprise, for example, adverse symptoms entered by the patient, abnormal signal readings from the sensors 201, or for merely transmitting personal information and patient history.

The control unit 102 communicates with the external monitoring station 103 using an in-built transceiver 102c as exemplarily illustrated in FIG. 5. The transceiver 102c transmits the patient information processed by the control unit 102 to the external monitoring station 103 by, for example, wired communication or wireless communication using short range and long range radio communication techniques. The processing of the patient information comprises, for example, converting the analog signals from the sensors 201 in the chest casing 101 into a digital form by analog to digital conversion. In an embodiment, the transceiver 102c may begin transmitting instantaneous patient information on the press of a transmit button on the user interface 102d, and continue transmitting signals until the press of a stop transmit button. The transceiver 102c acts as a communication bridge between the sensors 201 in the chest casing 101 and the external monitoring station 103 or the emergency medical service 104. The external monitoring station 103 communicates with the emergency medical services 104 for alerting the emergency medical services 104 of an emergency based on the patient information.

In an embodiment, the transceiver 102c in the control unit 102 comprises a global positioning system (GPS) that can be activated to locate the patient in an emergency situation. The GPS transmits information on the location of the patient to the external monitoring station 103 for alerting emergency medical services 104. The GPS may be activated automatically or manually by the patient by pressing an alarm button in the user interface 102d. The triangulated position of the patient is fed into the external monitoring station 103 or directly transmitted to the emergency medical service 104 or other emergency services.

The control unit 102 is also equipped with a speaker and optionally a microphone to provide audible instructions to the patient and to receive spoken utterances of the patient, respectively. The control unit 102 executes preloaded software for detecting, among other diagnoses, life-threatening situations by analyzing the signals from the sensors 201, such as a fast heart rate, ST segment elevation on the ECG indicating a heart attack, etc. In such situations, the control unit 102 may supply audio instructions to the patient such as to place an emergency call to an emergency medical service 104 or to rush to the nearest emergency room, etc. In order to detect abnormal signal variations, the control unit 102 measures and stores baseline patient information such as the name of the patient, age, gender, address, weight, height, medications, history of heart conditions and baseline ECG, baseline heart sounds, baseline lung sounds, baseline heart rate, blood pressure, temperature, oxygen saturation, baseline thoracic impedance, etc. The control unit 102 detects abnormal variations in the heart and cardiopulmonary conditions of the patient by comparing the baseline patient information with the collected patient information. In cases when the patient is incapable of responding to the instructions of the control unit 102 or initiate an emergency call based on physical symptoms experienced, the patient can signal the emergency medical service 104 by speaking instructions into the microphone. The microphone records the patient's utterances and transmits the recording to the emergency medical service 104 directly or via the external monitoring station 103. In non-emergency situations, the patient may wish to record any newly experienced symptoms using the microphone and transmit the recording to the external monitoring station 103 or a health care provider or professional.

In an embodiment, the external monitoring station 103 is, for example, a configured medical monitor or a patient monitoring system configured to receive and measure the patient information such as vital signs including ECG, heart sounds, lung sounds, blood pressure, pulse oxygenation, etc. The external monitoring station 103 may convert the patient information such as the ECG signals to a standard monitoring format for displaying the patient information or transmitting the patient information to the emergency medical service 104 or a health care center. The patient information transmitted to the health care center may be consulted by a health care professional before communicating suitable instructions back to the patient, treating physicians, or the emergency medical service team. The external monitoring station 103 may be remote to the patient or in the vicinity of the patient, or may comprise a network of two or more local and remote monitoring stations monitoring the patient information round the clock.

The system 100 disclosed herein further comprises one or more therapeutic devices 203 positioned on the chest casing 101 and interfaced with the control unit 102. In an embodiment, one or more external therapeutic devices are located in the vicinity of the patient and are capable of receiving instructions from the control unit 102 using wired and wireless interfacing techniques. The chest casing 101 comprises one or more therapeutic delivery points for delivering therapeutic electrical dosages to the patient automatically and/or based on feedback from the external monitoring station 103 to the control unit 102 in response to the patient information received at the external monitoring station 103. Therapeutic devices 203, for example, a pacemaker 203e, a defibrillator unit 203d, an oxygen storage unit 203c, an oxygen delivery apparatus 203a, a coolant circuit 203b, etc. as exemplarily illustrated in FIG. 2C and FIG. 5, are controlled by the control unit 102 to deliver therapeutic treatment to the patient. For example, the control unit 102 controls the pacemaker 203e to deliver electrical impulses to the patient through the therapeutic delivery points, for example, chest wall pads 202 of the chest casing 101 based on the feedback received from the external monitoring station 103. The control unit 102 automatically controls delivery of multiple therapies through the therapeutic delivery points on the chest casing 101 to the patient from the therapeutic devices 203 positioned on the chest casing 101 and/or in the vicinity of the patient.

A therapeutic addition to the system 100 may include a defibrillator unit 203d, situated in the vicinity of the patient, for shock delivery through the chest wall pads 202 with the approval of health care professionals. For example, the defibrillator unit 203d may be an automated external defibrillator (AED) that automatically diagnoses curable rhythms and delivers therapeutic electrical dosages to the patient to restore normal sinus rhythm. In an embodiment, the defibrillator unit 203d can be used as an external pacemaker 203e. The defibrillator unit 203d delivers electrical shocks to the patient in emergency conditions based on feedback instructions from the external monitoring station 103 via the control unit 102. The therapeutic delivery points in the chest casing 101 are, for example, in the form of peelable holes in the chest casing 101 or conductive strips with suitable gel to provide a low resistance interface for the chest wall pads 202 of the defibrillator unit 203d or the external pacemaker 203e with the patient's skin.

The control unit 102 also controls the delivery of oxygen from the oxygen storage unit 203c and by the oxygen delivery apparatus 203a based on the collected patient information and/or the feedback from the external monitoring station 103. The oxygen delivery apparatus 203a is, for example, an oxygen mask, a nasal cannula, etc. connected to the oxygen storage unit 203c via flexible tubing as exemplarily illustrated in FIG. 2C. In an embodiment, the oxygen storage unit 203c, the oxygen delivery apparatus 203a, and the coolant circuit 203b are positioned on the chest casing 101, in which case the oxygen delivery apparatus 203a and the coolant circuit 203b deliver cooling or oxygen therapy for a relatively short period of time. In another embodiment, the oxygen storage unit 203c and the coolant circuit 203b are connected to separate larger systems via tubes and valves to provide therapy for a longer period of time.

The control unit 102 receives signals from the sensors 201 in the chest casing 101 and transmits the processed information to the external monitoring station 103. In response, the external monitoring station 103 may diagnose a life-threatening arrhythmia based on the sensor signals, and communicates or transmits instructions back to the patient or the chest casing 101 via the control unit 102 to perform certain therapeutic functions such as administering electric dosages for restoring normal rhythm. The control unit 102 also controls an oxygen storage unit 203c and the oxygen delivery apparatus 203a to deliver therapeutic doses of oxygen based on the therapeutic feedback from the external monitoring station 103.

In an embodiment, the control unit 102 dynamically adjusts the automated delivery of one or more of the therapeutic electrical dosages and the cooling therapy in response to one or more of changes in the collected patient information, the heart and cardiopulmonary conditions, and instructions and feedback received from the external monitoring station 103. For example, the control unit 102 may gradually adjust the delivery of the therapeutic electrical dosages and the cooling therapy in response to changes in a subsequent batch of the collected patient information, which may indicate the patient's response to the ongoing therapeutic electrical dosages and/or the cooling therapy. Therefore, the control unit 102 dynamically adjusts the delivery of treatment based on the patient's response to the ongoing treatment and gradually restores normal cardiac rhythm or functionality.

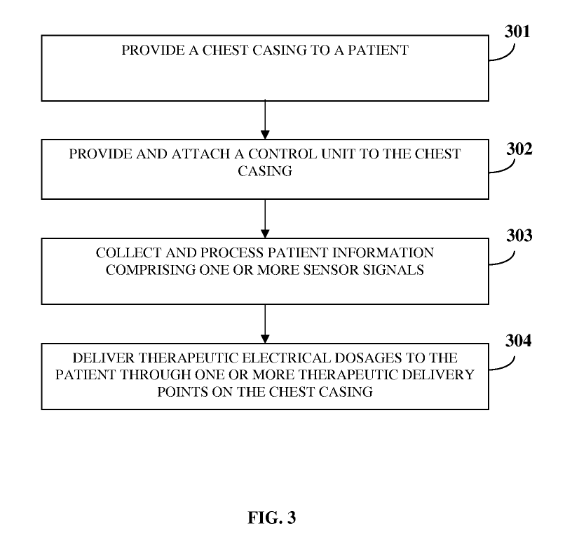

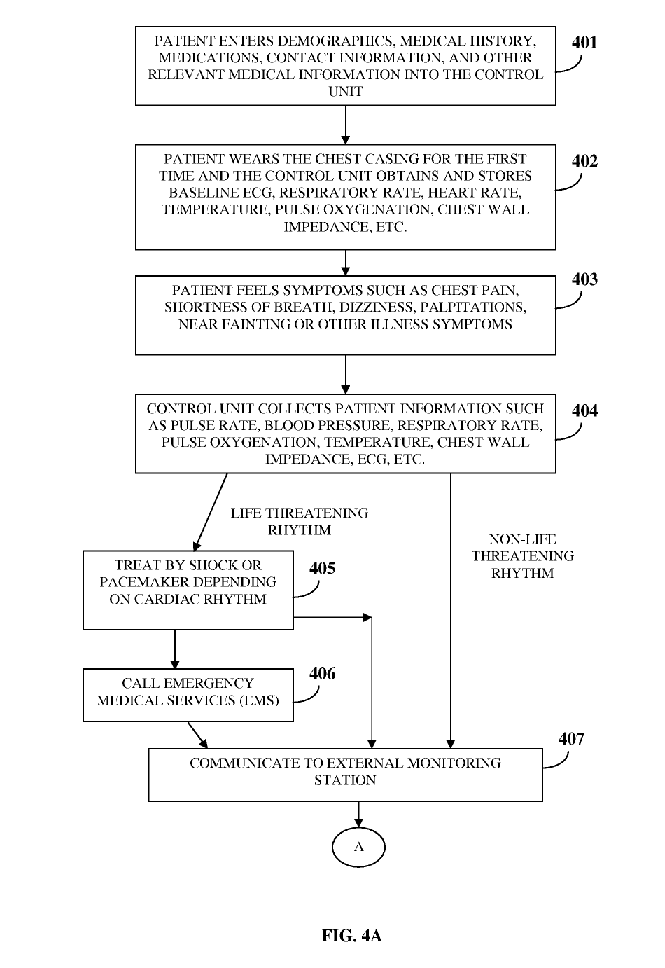

FIG. 3 illustrates a method for detection, forewarning, and rapid therapeutic treatment of heart and cardiopulmonary conditions in the patient. A chest casing 101, as disclosed in the detailed description of FIG. 1 and FIGS. 2A-2C, is provided 301 to the patient. The chest casing 101 is made of a form fitting material configured to conform to the patient's upper body. A control unit 102 is provided and attached 302 to the outer surface 101b of the chest casing 101 and connected to the sensors 201, for example, the electrocardiogram sensor electrodes 201c, the auscultation sensors 201d, the impedance sensor electrodes 201b, etc. Patient information comprising one or more sensor signals is collected and processed 303 by the control unit 102. For example, the control unit 102, in electronic communication with the impedance sensor electrodes 201b, processes the thoracic impedance measured across two or more points on the patient's chest wall to detect signs of congestive heart failure. The control unit 102 communicates with an external monitoring station 103 for transmitting the processed patient information to the external monitoring station 103. Therapeutic electrical dosages are delivered 304 to the patient through one or more therapeutic delivery points on the chest casing 101 automatically and/or based on feedback from the external monitoring station 103 to the control unit 102 in response to the transmitted patient information. The control unit 102 controls delivery of therapies, for example, cooling therapy, delivery of oxygen, etc. through the therapeutic delivery points on the chest casing 101 to the patient from one or more therapeutic devices 203, for example, the embedded coolant circuits 203b, the oxygen storage unit 203c, the oxygen delivery apparatus 203a, etc. positioned on and/or in the vicinity of the chest casing 101.

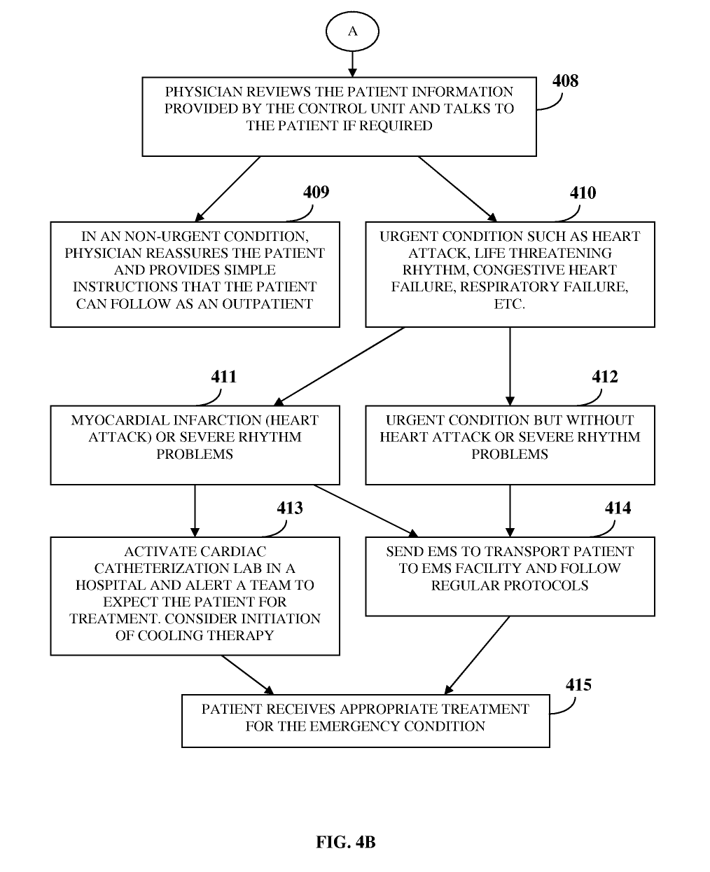

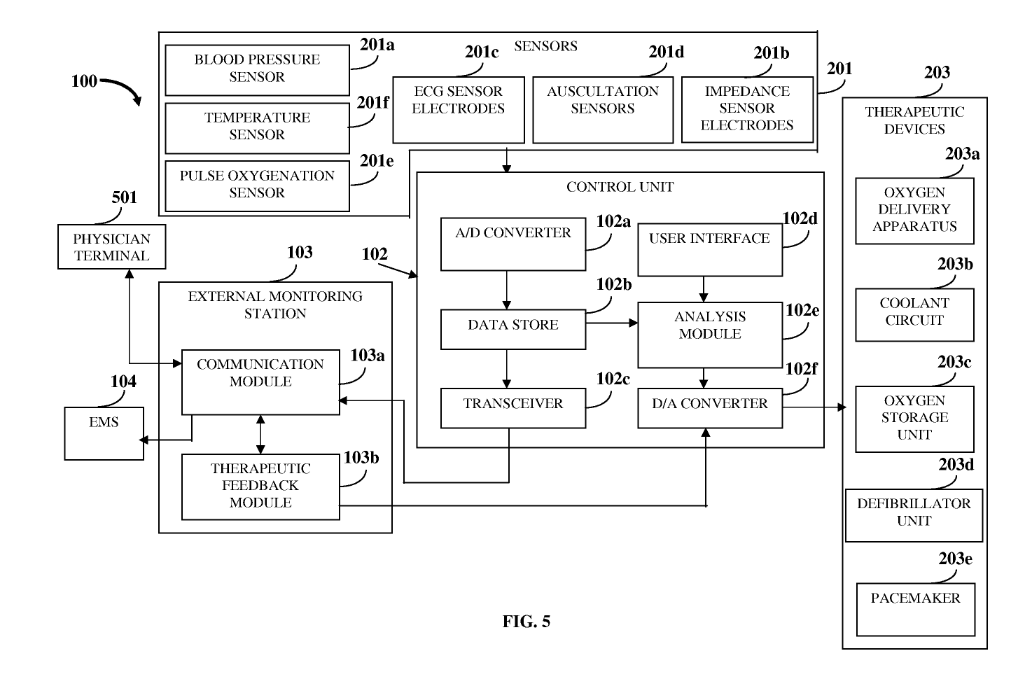

FIG. 4 exemplarily illustrates a flow diagram for detection, forewarning, and therapeutic treatment of a heart condition in the patient. When the patient obtains the chest casing 101, the patient enters 401 demographics, medical history, medications, contact information, and other relevant medical information into the control unit 102 via the user interface 102d. The patient wears 402 the chest casing 101 for the first time and the control unit 102 obtains and stores baseline patient information, for example, baseline ECG, respiratory rate, heart rate, temperature, pulse oxygenation, chest wall impedance or thoracic impedance, etc. After obtaining the baseline patient information, the patient removes the chest casing 101 for later use. Eventually, when the patient feels 403 symptoms such as chest pain, shortness of breath, dizziness, palpitations, near fainting or other illness symptoms, the patient wears the chest casing 101 and ensures that the power source 204 is connected to chest casing 101 and turned on. At this point, the control unit 102 collects 404 the patient information such as pulse rate, blood pressure, respiratory rate, pulse oxygenation, temperature, chest wall impedance, ECG, etc. from the corresponding sensors 201. The control unit 102 processes and transmits the collected patient information to the external monitoring station 103 to determine whether the sensor signals indicate abnormal variations in the heart condition. If an abnormal heart condition, for example, a life threatening cardiac rhythm is detected, the patient is treated 405 by automatically administering one or more timed electrical shocks or by administering electrical stimulus through the pacemaker 203e depending on the cardiac rhythm of the patient. The emergency medical service 104 is alerted about the emergency condition by placing a call 406 through the transceiver 102c in the control unit 102, and all the patient information is communicated 407 to the external monitoring station 103.

A medical professional, for example, a physician reviews 408 the patient information provided by the control unit 102 through the external monitoring station 103, and talks to the patient if required. The patient information is transmitted to a physician's terminal 501, as exemplarily illustrated in FIG. 5, over the internet or other communication networks. In a non-urgent condition, the physician reassures 409 the patient and provides simple instructions that the patient can follow as an outpatient. Based on the patient information, the physician may assess an urgent condition 410 such as heart attack, life threatening rhythm, congestive heart failure, respiratory failure, etc. In situations such as myocardial infarction or heart attack and severe rhythm problems 411, a cardiac catheterization lab is activated 413 in a hospital and a team is alerted by the external monitoring station 103 to expect the patient for treatment. At the same time, the initiation of the cooling therapy for the patient is considered and the coolant circuit 203b on the chest casing 101 is activated. The emergency medical service 104 is sent 414 to transport the patient to an emergency medical service facility or to the hospital and regular protocols are followed. In other urgent conditions without indications of heart attack or severe rhythm problems 412, the emergency medical service 104 is sent 414 to bring the patient to the emergency medical service facility and regular protocols are followed. The patient therefore receives 415 an appropriate treatment for the emergency condition.

FIG. 5 exemplarily illustrates an architectural block diagram of the system 100 for detection, forewarning, and rapid therapeutic treatment of heart and cardiopulmonary conditions in a patient. Multiple sensors 201, for example, a blood pressure sensor 201a, a temperature sensor 201f, a pulse oxygenation sensor 201e, the ECG sensor electrodes 201c, the auscultation sensors 201d, the impedance sensor electrodes 201b, etc. disposed on the chest casing 101 are interfaced to an analog to digital (A/D) converter 102a in the control unit 102 attached to the chest casing 101 to collect patient information in the form of sensor signals. The A/D converter 102a converts the analog sensor signals into the digital signals and feeds the digital signals into a data store 102b and an analysis module 102e. The digital signals representing the patient information are also transmitted to the external monitoring station 103 via the transceiver 102c. The communication module 103a in the external monitoring station 103 receives the transmitted patient information and relays the patient information to a physician terminal 501 and/or the emergency medical service (EMS) 104, if necessary. The user interface 102d on the control unit 102 comprises a keypad, a speaker, and a microphone for the patient to enter patient information, signal emergency conditions using the alarm button, or to converse with a health care professional using the transceiver 102c, etc.

The analysis module 102e compares the collected patient information with the previously captured baseline patient information to determine abnormal variations in the heart condition of the patient. If abnormal variations are detected in the heart condition of the patient, these variations are signaled to the external monitoring station 103, which in turn relays the emergency condition along with the associated patient information to the physician terminal 501 and the emergency medical service 104. Based on the detected variations, the therapeutic feedback module 103b of the external monitoring station 103 selectively instructs one or more therapeutic devices 203, for example, the defibrillator unit 203d, the pacemaker 203e, the oxygen storage unit 203c, the oxygen delivery apparatus 203a, the coolant circuit 203b, etc. via the digital to analog (D/A) converter 102f of the control unit 102 to deliver therapeutic treatment to the patient. In an embodiment, the therapeutic feedback module 103b instructs the control unit 102 to dynamically adjust the delivered therapeutic treatment in response to changes in the patient information. For example, the therapeutic feedback module 103b instructs the control unit 102 to dynamically adjust the delivered therapeutic treatment based on successive analyses performed by the analysis module 102e. The successive analysis performed by the analysis module 102e may indicate patient's response to the ongoing therapeutic treatment.

In certain life threatening rhythm abnormalities, for example, ventricular fibrillation, ventricular tachycardia or severe bradycardia, the control unit 102 is configured to deliver treatment automatically before receiving instructions from the external monitoring station 103, since these rhythms can lead to death in a very short time, for example, within a few minutes or seconds. The control unit 102 triggers the therapeutic devices 203 to automatically deliver therapeutic treatment to the patient. The control unit 102 may simultaneously communicate with the external monitoring station 103 regarding the ongoing rhythm abnormalities and the ongoing treatment. In response to the rhythm abnormalities and the ongoing treatment, the external monitoring station 103 can confirm, alter or stop further treatment depending on the clinical condition of the patient. This mechanism helps in rapid treatment during extreme emergencies and yet prevents errors in the delivery of the treatment.

The patient information relayed to the physician terminal 501 is reviewed by a health care professional to determine whether the patient is under a life threatening heart condition. The physician, at his/her discretion, may contact the patient via the external monitoring station 103 and the transceiver 102c in the control unit 102, and provide instructions to the patient. If a life threatening condition is indicated, the external monitoring station 103 may also alert the emergency medical service 104 via the communication module 103a.

The communication of the patient diagnostic information to the external monitoring station 103, which is supervised by a health care professional, enables the health care professional to analyze the sensor data, decide the level of urgency and provide appropriate instructions to the patient. In life threatening emergency situations, the control unit 102 issues an alert via the external monitoring station 103 to dispatch the emergency medical service 104 to the patient, and at the same time informs the nearest emergency department to expect the patient and have the available resources to treat the patient. For example, in case of acute myocardial infarction, the system 100 disclosed herein dramatically improves the outcome by instructing the emergency medical service team to administer blood thinners to the patient en route to the hospital, and at the same time prepares the cardiac catheterization team at the hospital to expect the patient for emergency angiography and angioplasty. This significantly reduces the door-to-balloon time, a national target, to less than the required 90 minutes, and also reduces the myocardial infarction-to-hospital time. Consequently, this improves the survival of patients with heart attacks, which is the most common cause of death in most countries.

The system 100 disclosed herein delivers emergency treatment to certain life threatening arrhythmias either by delivering shock to treat life threatening fast heart rhythms or by activating an external pacemaker 203e to correct cases of slow heart rate. This rapid therapeutic treatment can prevent fatal consequences till the emergency medical service 104 arrives to care for the patient. The automatic controlled therapeutic treatment provided by the system 100 disclosed herein is an important function since these cardiac rhythm problems can lead to the death of the patient in a few minutes.

Consider an example, where a healthy young patient develops a chest pain when the patient moves his arms. The patient wears the chest casing 101, and the control unit 102 attached to the chest casing 101 collects all the patient information, for example, ECG using the sensors 201 in the chest casing 101. The A/D converter 102a and the analysis module 102e in the control unit 102 process the patient information and the transceiver 102c transmits the patient information to the external monitoring station 103. The communication module 103a in the external monitoring station 103 receives the transmitted patient information and relays the patient information to a physician terminal 501. A health care professional reviews the patient information and determines that all the cardiac parameters and measurements are within normal ranges. The health care professional calls the patient to reassure the patient via the in-built transceiver 102c or by other means and instructed to follow up with the primary care physician (PCP) of the patient for musculoskeletal chest pain.

Consider another example, where a middle aged patient with a history of previous coronary angioplasty and stent develops mild chest pain. The patient wears the chest casing 101, and the control unit 102 attached to the chest casing 101 collects all the patient information, for example, ECG using the sensors 201 in the chest casing 101. The control unit 102 processes the patient information. The analysis module 102e in the control unit 102 analyzes the patient information to detect abnormal variations in the heart condition. The control unit 102 transmits the processed information to the external monitoring station 103 for subsequent review by a health care professional. A review of the ECG shows an ST elevation indicating a heart attack. The communication module 103a alerts the emergency medical service 104 about the emergency condition of the patient. The health care professional instructs the emergency medical service 104 to bring the patient to the nearest emergency room (ER) and administer blood thinners to the patient en route. The patient receives instructions to wait for the emergency medical service 104 via the in-built transceiver 102c and the speaker. In the meantime, the external monitoring station 103 activates the cardiac catheterization lab team to stand by at the hospital and rush the expected patient immediately to the catheterization lab, where an angiography of the patient reveals occluded artery. The occluded artery is subsequently opened by performing an angioplasty, following which the patient recovers from the heart attack.

Consider another example, where a senior patient with a history of heart attacks feels dizzy and shortness of breath. The patient wears the chest casing 101, and the control unit 102 attached to the chest casing 101 collects all the patient information, for example, ECG using the sensors 201 in the chest casing 101. The control unit 102 processes the ECG and other patient information to indicate ventricular tachycardia, that is, a life threatening fast rhythm. In an embodiment, the control unit 102 automatically delivers a shock to the patient through the chest wall pads 202 on the chest casing 101 using the defibrillator unit 203d. In another embodiment, the control unit 102 also transmits the processed information to the external monitoring station 103. In response to the transmitted patient information, the external monitoring station 103 instructs the control unit 102 to deliver a shock to the patient through the chest wall pads 202 on the chest casing 101 using the defibrillator unit 203d. The control unit 102 then alerts the emergency medical service 104 via the external monitoring station 103. The emergency medical service 104 is immediately dispatched to bring the patient to the hospital.

Consider another example, where a senior patient who had previously suffered a heart attack feels dizzy and shortness of breath. The patient wears the chest casing 101, following which the control unit 102 attached to the chest casing 101 collects all the patient information, for example, ECG using the sensors 201 in the chest casing 101. The control unit 102 processes the ECG and other patient information to indicate heart block with a junctional rhythm, that is, a life threatening slow rhythm. The control unit 102 transmits the processed information to the external monitoring station 103. In response to the transmitted patient information, the external monitoring station 103 instructs the control unit 102 to deliver external pacing through the chest wall pads 202 on the chest casing 101. The control unit 102 then alerts the emergency medical service 104 via the external monitoring station 103. The emergency medical service 104 is immediately dispatched to bring the patient to the hospital.

Consider another example, where a senior patient who is a heavy smoker develops a worsening condition of shortness of breath. The patient wears the chest casing 101, and the control unit 102 attached to the chest casing 101 collects all the patient information, for example, ECG, pulse oxygenation, etc. using the sensors 201 in the chest casing 101. The control unit 102 processes and transmits the ECG, pulse oxygenation and other patient information to the external monitoring station 103 for immediate review by a health care professional. The health care professional determines that the patient is severely hypoxic, for example, with the pulse oxygenation in the 70s. The patient is verbally instructed to suitably position the oxygen delivery apparatus 203a, for example, an oxygen mask or a nasal cannula. The oxygen storage unit 203c then starts delivering oxygen to the patient till the arrival of the emergency medical service (EMS) 104. The emergency medical service 104 is immediately dispatched to the patient, who is found suffering a respiratory failure. The emergency medical service 104 performs an emergency endrotracheal intubation and transfers the patient to the hospital.

Consider another example, where a senior patient with a history of previous heart attack starts developing a worsening condition of shortness of breath. The patient wears the chest casing 101 and the control unit 102 attached to the chest casing 101 collects all the patient information, for example, ECG, pulse oxygenation, and the thoracic impedance using the sensors 201 in the chest casing 101. The control unit 102 processes and transmits the ECG, pulse oxygenation, thoracic impedance and other patient information to the external monitoring station 103 for immediate review by a health care professional. The health care professional determines that the patient is severely hypoxic, for example, with the pulse oxygenation in the 80s. The thoracic impedance is found to be elevated compared to a baseline, indicating congestive heart failure. The patient is verbally instructed to suitably place the oxygen mask or nasal cannula. The oxygen storage unit 203c then starts delivering oxygen to the patient till the arrival of the EMS. The physician at the external monitoring station 103 may also instruct the EMS to administer intravenous diuretic to the patient when they arrive to start treating the congestive heart failure as early as possible.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

I claim:

1. A system for detection, forewarning, and rapid therapeutic treatment of heart and cardiopulmonary conditions in a patient, comprising:
a chest casing made of a form fitting material configured to conform to an upper body of said patient, wherein said chest casing defines an inner surface for establishing adequate contact with skin of said patient and an outer surface opposite to said inner surface, wherein said inner surface comprises:
a predefined plurality of electrocardiogram sensor electrodes for detecting abnormal electrocardiogram signals of said patient; and
a predefined plurality of auscultation sensors for recording internal sounds at predefined cardiac auscultation sensor points and respiratory auscultation sensor points located on a chest wall of said patient;
a control unit attached to said outer surface of said chest casing and connected to said electrocardiogram sensor electrodes and said auscultation sensors for collecting and processing patient information comprising one or more signals from said electrocardiogram sensor electrodes and said auscultation sensors, wherein said control unit communicates with an external monitoring station for transmitting said processed patient information to said external monitoring station; and
said chest casing comprising one or more therapeutic delivery points for delivering therapeutic electrical dosages to said patient automatically and/or based on feedback from said external monitoring station to said control unit in response to said transmitted patient information.

2. The system of claim 1, wherein said inner surface of said chest casing comprises one or more impedance sensor electrodes for measuring thoracic impedance across two or more points on said chest wall of said patient, wherein said control unit processes said measured thoracic impedance to detect signs of abnormality between said two or more points and to detect signs of congestive heart failure.

3. The system of claim 1, wherein said chest casing comprises one or more embedded coolant circuits for delivering cooling therapy to lower body temperature of said patient.

4. The system of claim 1, wherein said chest casing comprises one or more sleeves made of said form fitting material configured to conform to shoulders and arms of said patient, wherein an inner surface of said sleeves comprises one or more of a temperature sensor, a pulse oxygenation sensor, and a blood pressure sensor.

5. The system of claim 1, wherein said predefined cardiac auscultation sensor points on said chest wall of said patient for recording said internal sounds by said auscultation sensors correspond to apex of heart, aortic valve area, and left sternal border.

6. The system of claim 1, wherein said respiratory auscultation sensor points on said chest wall of said patient for recording said internal sounds by said auscultation sensors correspond to lower lung fields, middle lung fields, and upper lung fields on a back of said patient.

7. The system of claim 1, wherein said control unit stores baseline patient information comprising baseline electrocardiogram, baseline heart sounds, baseline lung sounds, baseline heart rate, blood pressure, temperature, oxygen saturation, and baseline impedance, wherein said control unit detects abnormal variations in said heart and cardiopulmonary conditions of said patient by comparing said baseline patient information with said collected patient information.

8. The system of claim 1, wherein said control unit dynamically adjusts delivery of one or more of said therapeutic electrical dosages and cooling therapy, in response to one or more of changes in said collected patient information, said heart and cardiopulmonary conditions, and instructions and said feedback received from said external monitoring station.

9. The system of claim 1, further comprising an oxygen storage unit and an oxygen delivery apparatus positioned on said chest casing for delivering oxygen to said patient, wherein said control unit controls said delivery of said oxygen by said oxygen storage unit and said oxygen delivery apparatus based on one or more of said collected patient information and said feedback from said external monitoring station.

10. The system of claim 1, further comprising chest wall pads positioned on said chest casing for shock delivery, wherein one or more of a defibrillator unit and a pacemaker connected on said chest wall pads automatically diagnoses curable rhythms and delivers said therapeutic electrical dosages to said patient to restore normal sinus rhythm.

11. The system of claim 1, wherein said control unit comprises a user interface for entering patient information, demographic information, and medical condition information by said patient and for receiving instructions and said feedback from said external monitoring station, and wherein said patient sends an alert to said external monitoring station via said user interface.

12. The system of claim 1, wherein said external monitoring station communicates with emergency medical services for alerting said emergency medical services of an emergency based on said transmitted patient information.

13. The system of claim 1, wherein said control unit comprises a global positioning system for transmitting information on location of said patient to said external monitoring station for alerting emergency medical services.

14. A system for detection, forewarning, and rapid therapeutic treatment of heart and cardiopulmonary conditions in a patient, comprising:
a chest casing made of a form fitting material configured to conform to an upper body of said patient, wherein said chest casing defines an inner surface for establishing adequate contact with skin of said patient and an outer surface opposite to said inner surface, wherein said inner surface comprises:
a predefined plurality of electrocardiogram sensor electrodes for detecting abnormal electrocardiogram signals of said patient;
a predefined plurality of auscultation sensors for recording internal sounds at predefined cardiac auscultation sensor points and respiratory auscultation sensor points located on a chest wall of said patient; and
one or more impedance sensor electrodes for measuring thoracic impedance across two or more points on said chest wall of said patient;
a control unit attached to said outer surface of said chest casing and connected to said electrocardiogram sensor electrodes, said auscultation sensors, and said impedance sensor electrodes for collecting and processing patient information comprising one or more signals from said electrocardiogram sensor electrodes, said auscultation sensors, and said impedance sensor electrodes, wherein said control unit communicates with an external monitoring station for transmitting said processed patient information to said external monitoring station; and
said chest casing comprising one or more therapeutic delivery points for delivering therapeutic electrical dosages to said patient automatically and/or based on feedback from said external monitoring station to said control unit in response to said transmitted patient information, wherein said control unit dynamically adjusts delivery of said therapeutic electrical dosages in response to one or more of changes in said collected patient information, said heart and cardiopulmonary conditions, and instructions and said feedback received from said external monitoring station.

15. The system of claim 14, wherein said control unit processes said measured thoracic impedance to detect signs of congestive heart failure.

16. The system of claim 14, wherein said chest casing comprises one or more embedded coolant circuits for delivering cooling therapy for regulating said body temperature of said patient.

17. The system of claim 14, wherein said control unit stores baseline patient information comprising baseline electrocardiogram, baseline heart sounds, baseline lung sounds, baseline heart rate, blood pressure, temperature, oxygen saturation, and baseline impedance, wherein said control unit detects abnormal variations in said heart and cardiopulmonary conditions of said patient by comparing said baseline patient information with said collected patient information.

18. The system of claim 14, further comprising one or more of a plurality of therapeutic devices positioned on said chest casing and/or in the vicinity of said patient and controlled by said control unit for delivering a plurality of therapies to said patient.

19. A method for detection, forewarning, and rapid therapeutic treatment of heart and cardiopulmonary conditions in a patient, comprising:
providing a chest casing made of a form fitting material configured to conform to an upper body of said patient, wherein said chest casing defines an inner surface for establishing adequate contact with skin of said patient and an outer surface opposite to said inner surface, wherein said inner surface comprises:
a predefined plurality of electrocardiogram sensor electrodes for detecting abnormal electrocardiogram signals of said patient; and
a predefined plurality of auscultation sensors for recording internal sounds at predefined cardiac auscultation sensor points and respiratory auscultation sensor points located on a chest wall of said patient;
providing a control unit attached to said outer surface of said chest casing and connected to said electrocardiogram sensor electrodes and said auscultation sensors;
collecting and processing patient information comprising one or more signals from said electrocardiogram sensor electrodes and said auscultation sensors, wherein said control unit communicates with an external monitoring station for transmitting said processed patient information to said external monitoring station; and
delivering therapeutic electrical dosages to said patient through one or more therapeutic delivery points on said chest casing automatically and/or based on feedback from said external monitoring station to said control unit in response to said transmitted patient information.

20. The method of claim 19, further comprising measuring thoracic impedance across two or more points on said chest wall of said patient using one or more impedance sensor electrodes positioned and connected on said inner surface of said chest casing, wherein said control unit is in electronic communication with said impedance sensor electrodes and processes said measured thoracic impedance to detect signs of congestive heart failure.

21. The method of claim 19, further comprising delivering cooling therapy to said patient to lower body temperature of said patient using one or more coolant circuits embedded in said chest casing.

22. The method of claim 19, further comprising measuring and storing baseline patient information comprising baseline electrocardiogram, baseline heart sounds, baseline lung sounds, baseline heart rate, blood pressure, temperature, oxygen saturation, and baseline impedance by said control unit, wherein said control unit detects abnormal variations in said heart and cardiopulmonary conditions by comparing said baseline patient information with said collected patient information.

23. The method of claim 19, further comprising dynamically adjusting delivery of one or more of said therapeutic electrical dosages and cooling therapy by said control unit, in response to changes in said collected patient information, said heart and cardiopulmonary conditions, and instructions and said feedback received from said external monitoring station.

24. The method of claim 19, further comprising communicating with emergency medical services for alerting said emergency medical services of an emergency based on said transmitted patient information.

25. The method of claim 19, wherein said control unit controls delivery of a plurality of therapies through said one or more therapeutic delivery points on said chest casing to said patient from one or more of a plurality of therapeutic devices positioned on said chest casing and/or in the vicinity of said patient.
